(19) United States
(12) Patent Application Publication
(10) Pub. No.: US 2020/0348281 A1
(43) Pub. Date: Nov. 5, 2020

(54) METHOD FOR THE DISCRIMINATION BETWEEN MARINE AND TERRESTRIAL SEDIMENTARY ENVIRONMENTS BY SELECTIVELY EXTRACTED EXCHANGEABLE STRONTIUM TO BARIUM RATIO FROM TERRIGENOUS CLASTIC SEDIMENTS

(71) Applicants: Nanjing Center of Geological Survey, China Geological Survey, Nanjing (CN); East China Normal University, Shanghai (CN)

(72) Inventors: Aihua Wang, Nanjing (CN); Zhanghua Wang, Shanghai (CN); Jiankun Liu, Nanjing (CN); Fei Zhang, Nanjing (CN); Hualing Li, Nanjing (CN); Haibo Huang, Nanjing (CN)

(73) Assignees: Nanjing Center of Geological Survey, China Geological Survey, Nanjing (CN); East China Normal University, Shanghai (CN)

(21) Appl. No.: 16/533,455

(22) Filed: Aug. 6, 2019

(30) Foreign Application Priority Data
Apr. 30, 2019 (CN) 201910361373.4

(51) Int. Cl. G01N 33/24; C22B 26/20; G01N 1/40; C22B 3/20

(52) U.S. Cl. CPC G01N 33/24; C22B 26/20; G01N 2001/4083; C22B 3/20; G01N 1/4044

(57) ABSTRACT

A method of selective extracting exchangeable strontium and barium from sediments, comprising, collecting a predetermined quantity of a sedimentary sample, removing biogenic clasts from the predetermined quantity of the sedimentary sample to provide a coarse filtered sedimentary sample, baking the coarse filtered sedimentary sample at a temperature less than 105° C., crushing the coarse filtered sedimentary sample to yield a fine filtered sedimentary sample having a sample grain size of less than 100 mesh, reacting a portion of the fine filtered sedimentary sample in a solution containing at least one of an ammonium acetate reactant and a sodium acetate reactant, measuring a strontium level within a liquid product of the solution, measuring a barium level within the liquid product of the solution and determining a ratio of the strontium level and the barium level of the liquid product.

### BACKGROUND

The present disclosure is generally related to selective extraction of exchangeable Strontium (Sr) and barium (Ba) from terrigenous clastic Sediments to distinguish between marine and terrestrial sedimentary environments of terrigenous clastic sediments.

Strontium (Sr) and barium (Ba) are alkaline earth metals and exhibit similar geochemical behaviors in endogenous geological processes. Due to differences in their chemical properties, they behave differently in exogenetic sedimentary processes. Because the differences in the geochemical environments between rivers and the sea (e.g., Eh, pH, salinity, and the concentrations of other ions), especially in estuaries where seawater and river water interact, due to the different geochemical behaviors of strontium and barium in the ionic state in water, the result is that terrestrial terrigenous clastic sediment is enriched in barium, whereas marine terrigenous clastic sediments are enriched in strontium. Generally, the strontium/barium ratio is less than 1.0 in terrestrial terrigenous clastic sediments, the strontium barium ratio is greater than 1.0 in marine terrigenous clastic sediments. Therefore, the strontium/barium ratio may be used to distinguish between marine and terrestrial sedimentary environments of terrigenous clastic sediments.

At present, many sedimentary scientists and sedimentary geochemists use the strontium/barium ratio obtained by whole-rock chemical analysis or bulk sample analysis of terrigenous clastic sediments to discriminate between marine and continental sedimentary environment, but the discrimination results may be unsatisfactory. The main reason is that the sample pretreatment methods used by most of the researchers in the analysis are inappropriate. Because the concentration of strontium and barium measured by whole-rock analysis has two parts: a part is from the clastic minerals reflecting geological background information of the material source regions (tectonic environment, rock type, weathering characteristics), and another part comes into being during sedimentary processes reflecting marine or terrestrial sedimentary geochemical environment changes (sedimentogenic strontium and barium).

Currently, sedimentologists and sedimentary geochemists primarily use two types of analytical methods to measure strontium and barium in clastic sediments. The first analytical method is X-ray fluorescence spectroscopy (XRF methods, including the pressed powder pellet method and fused glass bead method), the other method is inductively coupled plasma optical emission spectrometry or inductively coupled plasma mass spectrometry (ICP-OES or ICP-MS, abbreviated as ICP methods). The XRF methods involve crushing a bulk sample to 200-Meth (approximately 74 microns), pressing powder or fused glass, then measuring a bulk sample element content. The ICP methods involve crushing a bulk sample to 200-mesh (approximately 74 microns), digesting the elements in a solution, then measuring the concentrations of elements with an appropriate ICP instrument. The current most commonly used ICP methods for digesting geological samples are acid digestion (via hydrochloric acid, nitric acid, hydrofluoric acid, and perchloric acid) and fusion (lithium tetraborate or lithium metaborate fusion followed by dilute acid extraction). The techniques are whole-rock chemical analysis. Using the above analytical methods, the analytical results of modern sediments represent the total amounts of strontium and barium in the bulk samples. In order to distinguish sedimentary environments, the sedimentogenic strontium and barium contents of clastic sediments are sought, not the total strontium and barium contents. A new sample pretreatment method may selectively extract the sedimentogenic strontium and barium from terrigenous clastic sediments in order to use the strontium/barium ratio to distinguish between marine and terrestrial sedimentary environments.

In response to the above problems, the applicant had previously invented the “selective extraction of sedimentogenic strontium and barium in terrigenous clastic sediments” (US10151018B2)” for selective extraction of sedimentogenic strontium and barium from terrigenous clastic sediments and discrimination between marine and terrestrial sedimentary environment. Although the discrimination effect is much better than the traditional total amount method, it is found that when the patented technology is applied to sediments with high carbonate content (e.g. the Loess of China), the extracted strontium is higher than that of sediments due to the isomorphism of calcium and strontium, which makes the strontium/barium ratio in terrestrial sediments larger than 1.0, thus affecting the correct discriminant of marine and terrestrial sedimentary environment.

In one embodiment, a method of selective extracting exchangeable strontium and barium from sediments, comprising at least one of collecting a predetermined quantity of a sedimentary sample, removing biogenic clasts from the predetermined quantity of the sedimentary sample to provide a coarse filtered sedimentary sample, baking the coarse filtered sedimentary sample at a temperature less than 105° C., crushing the coarse filtered sedimentary sample to yield a fine filtered sedimentary sample having a sample grain size of less than 100 mesh (approximately 149 microns), reacting a portion of the fine filtered sedimentary sample in a solution containing at least one of an ammonium acetate reactant and a sodium acetate reactant, measuring a strontium level within a liquid product of the solution, measuring a barium level within the liquid product of the solution and determining a ratio of the strontium level and the barium level of the liquid product.

In another embodiment, a method of extracting strontium and barium from sediments, comprising at least one of crushing a sedimentary sample to yield a fine sedimentary sample, reacting a portion of the fine sedimentary sample in a solution containing at least one of an ammonium acetate reactant and a sodium acetate reactant, measuring a strontium level within a liquid product, measuring a barium level within the liquid product and determining a ratio of the strontium level and the barium level of the liquid product.

### BRIEF DESCRIPTION OF THE DRAWINGS

Embodiments of the disclosure will now be described, by way of example, with reference to the accompanying drawings, wherein:

FIG. 1 depicts a variation of the exchangeable strontium to barium ratio with salinity in artificial delta sediments.

FIG. 2 depicts an example chart of strontium and barium content in different sedimentary environments of borehole samples in the Yellow River Delta in accordance with an embodiment of the disclosure.

FIG. 3 depicts an example of variation characteristics of the strontium to barium ratios in different sedimentary environments of borehole samples in the Yellow River Delta in accordance with an embodiment of the disclosure.

### DETAILED DESCRIPTION

It may be readily understood that the components of the present application, as generally described and illustrated in the figures herein, may be arranged and designed in a wide variety of different configurations. Thus, the following detailed description of the examples of a method as represented in the attached figures is not intended to limit the scope of the application as claimed, but is merely representative of selected examples of the application.

The features, structures, or characteristics of the application described throughout this specification may be combined in a suitable manner in one or more examples. For example, the usage of the phrases example, examples, some examples, or other similar language, throughout this specification refers to the fact that a particular feature, structure, or characteristic described in connection with the example may be comprised in at least one example of the present application. Thus, appearances of the phrases example, examples, in some examples, in other examples, or other similar language, throughout this specification does not necessarily refer to the same group of examples, and the described features, structures, or characteristics may be combined in a suitable manner in one or more examples.

One objective of this new technique is to selectively extract the exchangeable strontium (Sr) and barium (Ba) from the total strontium and barium in multiple forms derived from multiple sources, thereby reducing the interference of non-sedimentogenic strontium and barium in silicate minerals and strontium and barium in material-derived detrital carbonates. Thus, this selective extraction may allow determination of the strontium level to barium level ratio to distinguish between marine and terrestrial sedimentary environments.

The strontium and barium in terrigenous clastic sediments may exist in many forms. They may occur in rock-forming minerals (including; K-feldspar, plagioclase, and amphibole and so on), in adsorbed forms or as ions in clay minerals formed by weathering, transport, and deposition processes, and in authigenic minerals formed in sedimentary processes. The strontium and barium may be exchangeable and carbonates bound, Fe—Mn oxides bound, organic and reduction materials bound, and residual (vast majority is silicate minerals) in terrigenous clastic sediments. Regardless of origin, source, and form of existence, the sedimentogenic strontium and barium are extracted from terrigenous clastic sediments for distinguishing between marine and continental sedimentary environments, because of the complexity of the genesis and occurrence of elements in sediments, it has been difficult to achieve reliably. It may be possible to reduce the interference of non-sedimentogenic strontium and barium unrelated to sedimentary environments, the selective extraction method of the present disclosure provides one such reliable method.

One technical issue that this method may solve is finding a reliable extraction agent and method for extracting the exchangeable strontium and barium, which reflect the characteristics of the sedimentary environment, from the multi-genesis, multi-source, and multi-occurrence form strontium and barium in terrigenous clastic sediments.

Previous studies have found that approximately 50% of the strontium in terrigenous clastic sediments is present in silicate rock-forming minerals (including K-feldspar, plagioclase, and amphibole and so on), and that approximately 40% is in exchangeable forms and bound to carbonates. More than 80% of the barium is present in silicate rock-forming minerals (including K-feldspar, plagioclase, and amphibole and the like), and less than 10% is in exchangeable forms and bound to carbonates or Fe—Mn oxides. Silicate rock-forming minerals, including K-feldspar, plagioclase, and amphibole and so on, are generally not digested by acids other than hydrofluoric acid. This property allows the selective extraction of sedimentogenic strontium and barium (Mainly exchangeable and bound to carbonate) from clastic sediments and reducing interfere of non-sedimentogenic strontium and barium related to the provenance. Therefore, the applicant previously invented the patented technology for the extraction of Sedimentogenic strontium and barium from terrigenous clastic sediments (US10151018B2).

Further research also found that the carbonate in terrigenous clastic sediments is syn-sedimentogenic and material-derived detrital. However, strontium and barium present in material-derived detrital carbonates may also interfere with the discrimination of marine and terrestrial sedimentary environments. And it is found that there is a good linear relationship between the exchangeable strontium/barium ratio and salinity by experiment.

To use the strontium/barium ratio to preferably discriminate between marine and terrestrial sedimentary environment of terrigenous clastic sediments, multiple agents, and their combinations were tested to obtain a reliable selective extraction agent and method to extract exchangeable strontium and barium.

A predetermined quantity of representative terrigenous clastic sediments is collected, visible biogenic clasts (shells, etc.) are removed, the sample is baked at a low temperature, i.e. less than 105 degrees Celsius, then crashed to a grain size no larger than 100-mesh (approximately 149 microns) to yield a fine filtered sedimentary sample. The sample is weighed and an ammonium acetate reactant or sodium acetate reactant is added with a 5-50% or 2-20% mass percentage concentration respectively as an extraction agent in 1:50-1:500 solid-liquid ratio. The sample and solution are stirred or oscillated (ultrasonic oscillation or mechanical oscillation) at room temperature (20-30° C.) and normal pressure for more than 120 minutes. The solid and liquid are separated, and the supernatant is diluted to an appropriate concentration for the instrumental analysis (by ICP-OES or ICP-MS) of strontium and barium. The gained strontium level to barium level ratio reflects whether the sediment was deposited in a marine or a terrestrial sedimentary environment.

Compared to whole-rock chemical analysis methods and licensed patent methods, the present method uses a more reliable extraction agent and method that extracts exchangeable strontium and barium from terrigenous clastic sediments in order to better distinguish between marine and terrestrial sedimentary environments. This method avoids the interference of non-sedimentogenic strontium mainly in silicate minerals (which can represent more than 50% of the total amount) and non-sedimentogenic barium mainly in silicate minerals (which can represent more than 80% of the total amount), and non-sedimentogenic strontium in material-derived detrital carbonates and non-sedimentogenic barium in material-derived detrital carbonates in clastic sediments. It thereby increases the effectiveness and accuracy of using the determined strontium/barium ratio to distinguish between marine and terrestrial sedimentary environments and may solve a basic theoretical problem that has troubled the fields of sedimentology and sedimentary geochemistry. Particularly, this analytical measurement method is simple, fast and reliable.

Based on the disclosed method, the following examples are presented to illustrate and explain various embodiments, but not to limit its range of applications.

### EXAMPLE 1

The selective extraction method for exchangeable strontium and barium in terrigenous clastic sediments includes the following steps. A predetermined quantity of representative loose sediment is collected, and visible biogenic clasts (shells, etc.) are removed to provide a coarse filtered sedimentary sample. The sample is baked at a low temperature, i.e. less than 105 degrees Celsius for twelve hours or greater duration and crushed to a grain size no larger than 100-mesh, (approximately 74 microns) to yield a fine filtered sedimentary sample. A portion of the sample is weighed, and an ammonium acetate solution or sodium acetate solution with a 20% mass percentage concentration added as an extraction agent at a 1:50 solid-liquid ratio. The mixture is stirred or oscillated (ultrasonic oscillation or mechanical oscillation) at room temperature (20-30° C.) and normal pressure for more than 120 minutes, and the solid and liquid are separated. The supernatant is diluted to an appropriate concentration for the instrumental analysis (by ICP-OES or ICP-MS) of the strontium and barium concentrations. The gained strontium/barium ratio reflects whether the sample was deposited in a marine or terrestrial sedimentary environment.

### EXAMPLE 2

The selective extraction method for exchangeable strontium and barium in terrigenous clastic sediments includes the following steps. A predetermined quantity of representative loose sediment is collected, and visible biogenic clasts (shells, etc.) are removed to yield a coarse filtered sedimentary sample. The sample is baked at a low temperature, less than 105 degrees Celsius and crushed to a grain size no larger than 100-mesh, (approximately 74 microns). A portion of the sample is weighed, and an ammonium acetate solution or sodium acetate solution with a 15% mass percentage concentration added as an extraction agent at a 1:100 solid-liquid ratio. The mixture is stirred or oscillated (ultrasonic oscillation or mechanical oscillation) at room temperature (20-30° C.) and normal pressure for more than 120 minutes, and the solid and liquid are separated. The supernatant is diluted to an appropriate concentration for the instrumental analysis (by ICP-OES or ICP-MS) of the strontium and barium concentrations. The gained strontium/barium ratio reflects whether the sample was deposited in a marine or terrestrial sedimentary environment.

### EXAMPLE 3

The selective extraction method for exchangeable strontium and barium in terrigenous clastic sediments includes the following steps. A predetermined quantity of representative loose sediment is collected, and visible biogenic clasts (shells, etc.) are removed. The sample is baked at a low temperature, less than 105 degrees Celsius for approximately twelve hours or greater duration, and crushed to a grain size no larger than 100-mesh, (approximately 74 microns). A portion of the sample is weighed, and an ammonium acetate solution or sodium acetate solution with a 10% mass percentage concentration added as an extraction agent at a 1:200 solid-liquid ratio. The mixture is stirred or oscillated (ultrasonic oscillation is also an option) at room temperature (20-30° C.) and normal pressure for more than 120 minutes, and the solid and liquid are separated. The supernatant is diluted to an appropriate concentration for the instrumental analysis (by ICP-OES or ICP-MS) of the strontium and barium concentrations. The gained strontium/barium ratio reflects whether the sample was deposited in a marine or terrestrial sedimentary environment.

### EXAMPLE 4

The selective extraction method for exchangeable strontium and barium in terrigenous clastic sediments includes the following steps. A predetermined quantity of representative loose sediment is collected, and visible biogenic clasts (shells, etc.) are removed. The sample is baked at a low temperature, i.e. 105 degrees Celsius, and crushed to a grain size no larger than 100-mesh, (approximately 74 microns). A portion of the sample is weighed, and an ammonium acetate solution or sodium acetate solution with a 5% mass percentage concentration added as an extraction agent at a 1:300 solid-liquid ratio. The mixture is stirred or oscillated (ultrasonic oscillation or mechanical oscillation) at room temperature (20-30° C.) and normal pressure for more than 120 minutes, and the solid and liquid are separated. The supernatant is diluted to an appropriate concentration for the instrumental analysis (by ICP-OES or ICP-MS) of the strontium and barium concentrations. The gained strontium/barium ratio reflects whether the sample was deposited in a marine or terrestrial sedimentary environment.

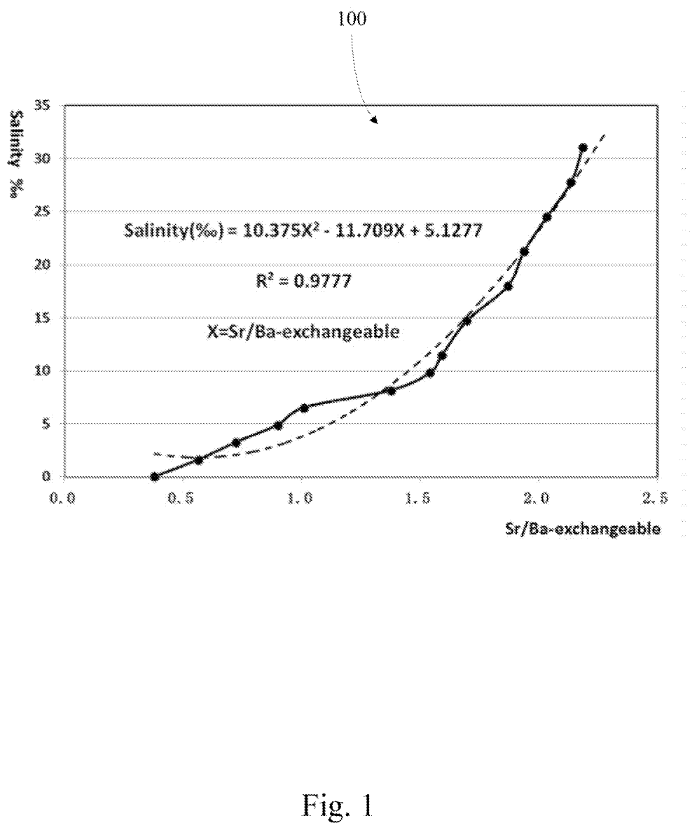

FIG. 1 shows the variation of the exchangeable strontium/barium ratio with salinity in artificial delta sediments. The experimental results show that with the increase of water salinity, the exchangeable strontium/barium ratio in the sediments increases gradually.

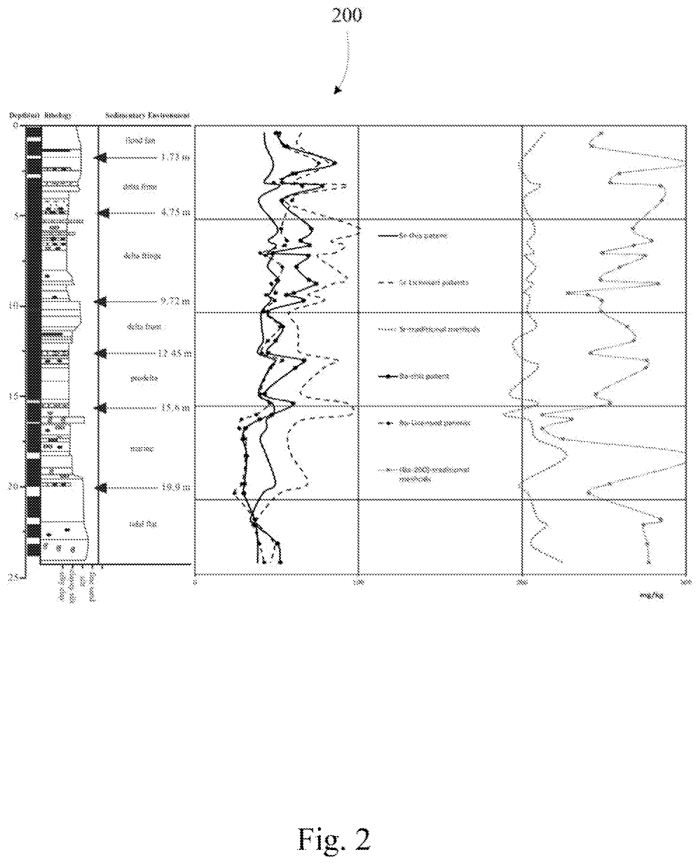

FIG. 2 is a characteristic chart of strontium and barium content in different sedimentary environments of borehole samples in the Yellow River Delta measured in accordance with an embodiment of the disclosure, authorized patent method and traditional method. Because the sediments of the Yellow River Delta originating from the loess of China contain a large amount of carbonate, the strontium extracted by 10% acetic acid of the licensed patented method is obviously higher than that extracted by 1MNH4Ac method of this disclosure, and the barium extracted by the licensed patented method is significantly lower than that extracted by disclosed embodiments described within this disclosure (the barium extracted by the traditional method in the figure is reduced by 200 mg/kg from the measured value, so the “(Ba-200)-traditional methods” is expressed).

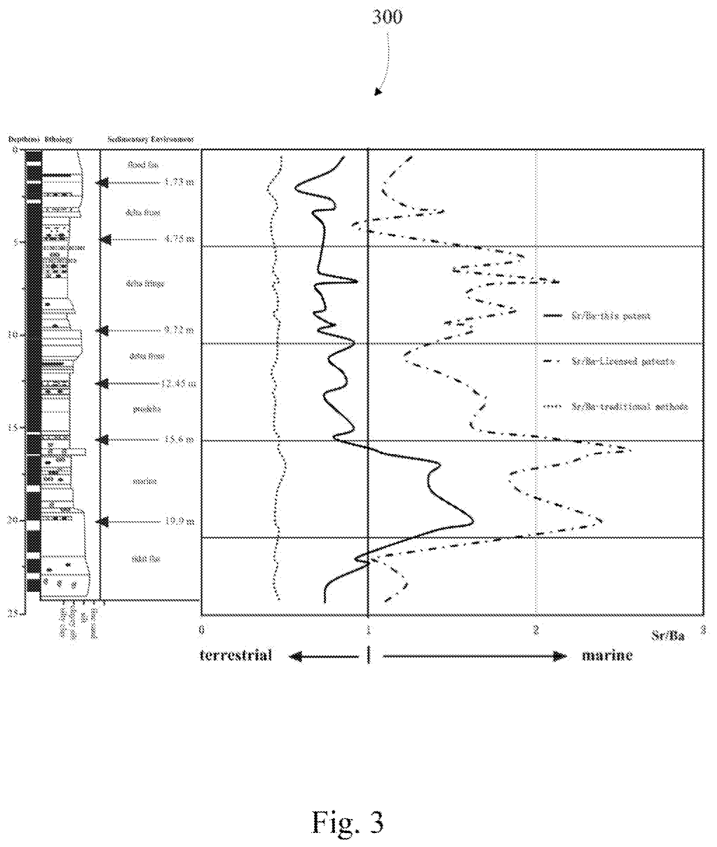

FIG. 3 shows the variation characteristics of the strontium/barium ratios in different sedimentary environments of borehole samples in the Yellow River Delta obtained by embodiments described within this disclosure, licensed patented method and traditional method. Although the resolution of the authorized patented method has been improved, it does not conform to the rule that the common terrestrial sedimentary environment is less than 1.0 (because of the interference of high content of strontium extracted by dilute acetic acid in detrital carbonate due to the isomorphism of Strontium-Calcium carbonate minerals). The disclosed embodiments reducing the interference of material-derived strontium and ensures that the strontium/barium ratio of marine sediments is greater than 1.0.

Although exemplary examples the method of the present disclosure have been illustrated in the accompanied drawings and described in the foregoing detailed description, it will be understood that the application is not limited to the examples disclosed, and is capable of numerous rearrangements, modifications, and substitutions without departing from the spirit or scope of the disclosure as set forth and defined by the following claims.

The above examples are for illustrative purposes and are not intended to limit the scope of the disclosure or the adaptation of the features described herein to particular components. Those skilled in the art will also appreciate that various adaptations and modifications of the above-described preferred examples may be configured without departing from the scope and spirit of the disclosure. Therefore, it is to be understood that, within the scope of the appended claims, the disclosure may be practiced by examples in addition to those specifically described.

What is claimed is:

1. A method of selective extracting exchangeable strontium and barium from sediments, comprising:
collecting a predetermined quantity of a sedimentary sample;
removing biogenic clasts from the predetermined quantity of the sedimentary sample to provide a coarse filtered sedimentary sample;
baking the coarse filtered sedimentary sample at a temperature less than 105° C.;
crushing the coarse filtered sedimentary sample to yield a fine filtered sedimentary sample having a sample grain size of less than 100 mesh;
reacting a portion of the fine filtered sedimentary sample in a solution containing at least one of an ammonium acetate reactant and a sodium acetate reactant;
measuring a strontium level within a liquid product of the solution;
measuring a barium level within the liquid product of the solution; and
determining a ratio of the strontium level and the barium level of the liquid product.

2. The method of claim 1 wherein the ratio of the strontium level to the barium level is determined.

3. The method of claim 1 wherein the ratio of the barium level to the strontium level is determined.

4. The method of claim 1 wherein the solution is comprised of at least one of between 5% and 50% the ammonium acetate reactant and between 2% and 20% the sodium acetate reactant.

5. The method of claim 1 wherein the solution is a 1M solution of at least one of the ammonium acetate reactant and the sodium acetate reactant.

6. The method of claim 1 wherein solid-liquid ratio of the portion of the fine filtered sedimentary sample and an extraction agent of the solution is between 1:50-1:500.

7. The method of claim 1 wherein the sample grain size of the fine filtered sedimentary sample is less than 150 microns.

8. The method of claim 1 wherein the baking is at least twelve hours in duration.

9. The method of claim 1 further comprising at least one of stirring and oscillating the solution for at least 120 minutes.

10. The method of claim 9 wherein the oscillating is at least one of mechanical and ultrasonic.

11. A method of extracting strontium and barium from sediments, comprising:
crushing a sedimentary sample to yield a fine sedimentary sample;
reacting a portion of the fine sedimentary sample in a solution containing at least one of an ammonium acetate reactant and a sodium acetate reactant;
measuring a strontium level within a liquid product;
measuring a barium level within the liquid product; and
determining a ratio of the strontium level and the barium level of the liquid product.

12. The method of claim 11 wherein the ratio of the strontium level to the barium level is determined.

13. The method of claim 11 wherein the ratio of the barium level to the strontium level is determined.

14. The method of claim 11 wherein the solution is comprised of at least one of 5% to 50% the ammonium acetate reactant and 2% to 20% the sodium acetate reactant.

15. The method of claim 11 wherein the solution is a 1M solution of at least one of the ammonium acetate reactant and the sodium acetate reactant.

16. The method of claim 11 wherein a solid-liquid ratio of the fine sedimentary sample and an extraction agent of the solution is between 1:50-1:500.
